(19) United States
(12) Patent Application Publication
(10) Pub. No.: US 2007/0092583 A1
(43) Pub. Date: Apr. 26, 2007

(54) Cold remedy composition comprising zinc salts

(76) Inventor: Tim Clarot, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 10/556,738

(22) PCT Filed: Apr. 30, 2004
(86) PCT No.: PCT/US04/13388

§ 371 Date: Nov. 13, 2006

Related U.S. Application Data
(60) Provisional application No. 60/466,956, filed on Apr. 30, 2003.

(51) Int. Cl. A61K 33/32; A61K 31/315

(52) U.S. Cl. 424/643; 514/494

(57) ABSTRACT

A composition to treat symptoms of cold and/or prevent the onset of cold symptoms and methods of forming and using the composition are disclosed. The composition is formulated to maintain one or more active ingredients in association with an oral membrane for an extended period of time. The invention further includes a method for applying the composition to the oral membrane.

### FIELD OF THE INVENTION

The present invention relates generally to a composition for reducing symptoms associated with colds. More particularly, the invention relates to a tablet composition that includes one or more active ingredients for treating symptoms associated with colds and to methods of using and forming the composition.

### BACKGROUND OF THE INVENTION

Millions of people each year suffer from colds, which are caused by a variety of viruses. Symptoms typically associated with colds include headaches, muscle aches, coughing, fever, chills, runny nose, sore throat, watery eyes, diarrhea, and vomiting. Because the symptoms can be quite severe and the viruses that cause the cold symptoms are easily spread to others—particularly via sneezing and the like—it is desirable to have fast-acting relief to apply at the early onset of such symptoms and/or compositions that prevent the onset of the symptoms.

Typical cold relief remedies are administered orally and distributed to the blood system via the digestive system Although such remedies may work well for relieving some cold symptoms, the effectiveness of many of these remedies may be limited due to, for example, digestive processes in the oral and digestive pathways. For example, enzymatic activity in the oral cavity and/or acidic environments present in the digestive system may degrade the performance of specific elements or compounds comprising active substances in cold relief compositions. An additional problem associated with typical cold remedies is that several people, particularly young children and older adults, have problems ingesting orally administered remedies.

Accordingly, a fast-acting composition for treating cold symptoms and/or preventing the onset of the symptoms and methods of using and forming the compositing are desired.

### SUMMARY OF THE INVENTION

The present invention provides improved methods and compositions for treating symptoms associated with colds. While the ways in which the present invention addresses the deficiencies and disadvantages of the prior art are described in greater detail hereinbelow, in general, according to various aspects of the present invention, a method and composition are provided for quickly delivering relief to cold sufferers and/or those wishing to prevent the onset of cold symptoms.

In accordance with various embodiments of the present invention, a cold remedy composition is formulated to permit the active substance to remain in contact with an oral membrane for an extended period of time. Maintaining the active ingredients in contact with the oral membrane for an extended period of time facilitates absorption of the active ingredient through the membrane, which in turn increases the speed of symptom relief

In accordance with various embodiments of the invention, a composition for application to a oral membrane is configured to maintain an active substance, such as metallic and/or ionic zinc, in contact with the oral membrane for an amount of time sufficient to permit delivery of a desired amount of the active substance to active sites on the oral membrane, and in further embodiments, across the oral membrane into the blood and/or circulatory system of a patient. In accordance with various aspects of this embodiment, the active ingredients include a homeopathic amount of the active agent. In accordance with further aspects, the active ingredients include an allopathic agent.

In accordance with various other embodiments of the invention, the composition is formed as a dissolvable tablet configured to disintegrate or dissolve within an oral cavity, while maintaining the active substance in contact with the oral cavity for an extended period of time. This aspect of the invention is advantageous over the prior art because it does not require swallowing of a liquid or a pill, which can be objectionable to some people.

In accordance with another embodiment of the invention, a composition for treating a cold symptom is formed by admixing one or more active ingredients, a bulk or inert agent, and a dissolution agent. The composition is formed into tablets suitable for oral application of the active ingredient.

In accordance with yet another embodiment of the invention, a method of using the cold remedy composition includes applying a desired amount of the composition to an oral membrane. In accordance with various aspects of this embodiment, a dose of one tablet is administered to a patient, no more frequently than about every three to four hours, up to about 48 hours after the symptoms have subsided.

### DETAILED DESCRIPTION

In accordance with various aspects of the present invention, a composition is provided, which includes an inert substance and one or more active substances. The composition is formulated to promote maintenance of the active substance in direct contact with at least a portion of the oral cavity for an extended period of time. As described below, the composition may also include additional ingredients such as texture agents, dissolution agents, buffers, stabilizers, preservatives, sweeteners, and other taste modifiers.

As used heroin, an active substance includes any of one or more substances that produces or promotes a beneficial therapeutic, physiological, homeopathic, allopathic and/or pharmalogical effect on the body. Such beneficial effects may be brought upon any animal or human patient, and various systems associated therewith, including the immune system, respiratory system, circulatory system, nervous system, digestive system, urinary system, endocrine system, muscular system, skeletal system, and the like, as well as any organs, tissues, membranes, cells, and subcellular components associated therewith.

As will be appreciated by those skilled in the art, beneficial effects include assisting the more efficient functioning of the various systems described above, such as, for example, helping the body fight sickness and disease, helping the body to heal, etc. Exemplary active substances include any element, composition or material producing a beneficial effect, including vitamins, minerals, nucleic acids, amino acids, peptides, polypeptides, proteins, genes, mutagens, antiviral agents, antibacterial agents, anti-inflammatory agents, decongestants, histamines, herstamines, anti-histamines, anti-allergens, allergy-relief substances, homeopathic substances, pharmaceutical substances, and the like.

Exemplary active substances include metallic and ionic zinc, which is thought to bind to ICAM receptors within the oral cavity to inhibit the spread of the virus. When a composition comprising zinc is applied to the oral cavity, zinc ions from the composition adhere to a portion of the membrane in the oral cavity. It is believed that the zinc in the mucous or mucous membrane creates a barrier which inhibits viral infection of the oral membrane.

A homeopathic concentration of zinc ions in the zinc composition of the invention is about 0.5× to about 2×, preferably about 1× zinc gluconate and about 1× to about 3× zinc acetate.

The inert agent is configured to facilitate tablet formation and to maintain the active ingredient in contact with an oral membrane for an extended period of time. In accordance with various embodiments of the invention, the inert material includes purified talc.

The composition may also include a thickener, such as food-grade or pharmaceutical grade thickeners, including, for example, glycerin, carrageenan, sugar, guar gum, methylcellulose, aloe vera, cellulose (e.g. microcrystalline cellulose powder available from American International Chemical Inc. (AIC) under the brand name Emcocel 50), and the like.

The composition may also include other antiseptics, preservatives, permeation enhancers, sequestering agents, buffers, emulsifiers, or similar ingredients.

Exemplary texturing agents include colloidal silicon dioxide, crosspovidone magnesium stearate, and sorbitan monostearate. Compositions of the present invention may include any combination of these and/or other texturing agents.

Compositions of the present invention may also include dissolution agents to facilitate rapid dissolution of the tablet within the oral cavity. Exemplary dissolution agents include sodium lauryl sulphate, sodium starch glycolate, and polyvinyl pyrolidone.

In accordance with another aspect of the invention, a preservative may be added to the composition to facilitate stability of the various ingredients. Any suitable preservative may be used in accordance with the present invention. Suitable exemplary preservatives for use with the present invention include polysorbate 80.

To make the composition taste better, the composition may include sweeteners (real or artificial) and/or flavoring agents (real or artificial). For example, the composition may include artificial sweeteners such as manitol powder and/or sucralose powder, and a flavoring agent such as dried cherry flavor spray.

In accordance with various embodiments of the present invention, the composition is a homeopathic, allopathic, or pharmaceutical composition, which includes from about 2% to about 10% by weight of at least one active substance. The effective amount of the active substance includes any amount of active substance required in a composition or dose suitable to render a beneficial therapeutic effect. For example, in accordance with one aspect of this embodiment, the composition includes from about 1% to about 5% by weight zinc acetate dehydrate and from about 1% to about 5% zinc gluconate.

As noted above, the composition may also include permeation enhancers, which are believed to function by enlarging or loosening tight junctions between cells in the oral membrane, thereby facilitating passage of the active substance therethrough. Exemplary permeation enhancers include liposomes, sequestering agents, ascorbic acid (Vitamin C), glycerol, chitosan, and lysophosphotittylcholin, or any other substance that provides a similar function or result. By way of example, the permeation enhancer may include a sequestering agent, such as EDTA. EDTA is thought to chelate calcium. When applied to the oral membrane, it is believed to remove calcium from the cell junctions, thereby loosening the junctions to facilitate passage of an active substance therethrough.

The composition of the present invention may be delivered to the oral cavity according to any suitable method, such as administering a tablet or a powder form of the composition. In accordance with a preferred embodiment of the present invention, the composition is delivered as a single tablet. The method includes the steps of obtaining a composition in accordance with the present invention for delivery into the oral cavity. The method further includes the step of applying the composition in the oral cavity.

The composition may be delivered to the patient in any suitable dosage. Preferably, the composition is delivered to a patient about every three to four hours until desired dissipation of the symptoms is achieved and up to 48 hours after such dissipation.

### EXAMPLES

The Examples set forth hereinbelow are illustrative of various aspects of certain preferred embodiments of the present invention. The compositions, methods and various parameters reflected therein are intended only to exemplify various aspects and embodiments of the invention, and are not intended to limit the scope of the claimed invention.

### Example 1

An exemplary gel composition for relieving cold symptoms is prepared by admixing the following ingredients.

| | | | | Quantity | | Homeopathic |
|---|---|---|---|---|---|---|
| Generic Name | Brand Name | Grade | Manufacturer | (wt. %) | Range | Conc. |
| Zinc Acetate Dihydrate | | USP/ACS | American International | 3.07 | | |
| | | | Chemical Inc. (AIC) | | | |
| Zinc Gluconate | | USP/NF | American International | 2.36 | | |
| | | | Chemical Inc. (AIC) | | | |
| Mannitol Powder | Mannidex10700 | USP | Cerester | 58.13 | | |
| Microcrystalline | Emcocel50 | NF/BP/JP | Penwest | 22.08 | | |
| cellulose powder | | | | | | |
| Povidone (Polyvinyl | Plasdone | USF | ISP | 0.73 | | |
| Pymolidone k-29/32) | k-29/32 | | | | | |
| Sodium Starch Glycolate | Explotab | NF/BP | Penwest | 3.44 | | |
| Polysorbate-80 | Tween 80K | NF | ICI | 0.26 | | |
| Corrspovidone | Plasdone XL-10 | NF | ISP | 5.00 | | |
| Sodium lauryl sulphate | Stepanol WA-100 | HF | Stepan | 0.05 | | |
| Sorbitan Monostearate | Sorbitan 60K | Food | ADM arkady | 3 | | |
| Magnesium stearate | | USP | whittakar | 0.6 | | |
| Purified Talc | | USP/NF | whittakar | 0.2 | | |
| Colloidal silicon dioxide | Syfloid 244F | USP/NF | Grace | 0.1 | | |
| Cherry flavor Spray dried | | | David Michael & Co. | 2.5 | | |
| Micronlsed Sucralose Powder | Splenda | NF | Mcneil | 0.2 | | |

### Example 2

Another exemplary composition for relieving cold symptoms is prepared by admixing the following ingredients in the manner described above.

| | | | | Quantity | | Homeopathic |
|---|---|---|---|---|---|---|
| Generic Name | Brand Name | Grade | Manufacturer | (wt. %) | Range | Conc. |
| Zinc Gluconate | | USP/NF | American International | 5.43 | | |
| | | | Chemical Inc. (AIC) | | | |
| Mannitol Powder | Mannidex10700 | USP | Cerester | 58.13 | | |
| Microcrystalline | Emcocel50 | NF/BP/JP | Penwest | 22.08 | | |
| cellulose powder | | | | | | |
| Povidone (Polyvinyl | Plasdone | USF | ISP | 0.73 | | |
| Pymolidone k-29/32) | k-29/32 | | | | | |
| Sodium Starch Glycolate | Explotab | NF/BP | Penwest | 3.44 | | |
| Polysorbate-80 | Tween 80K | NF | ICI | 0.26 | | |
| Corrspovidone | Plasdone XL-10 | NF | ISP | 5.00 | | |
| Sodium lauryl sulphate | Stepanol WA-100 | HF | Stepan | 0.05 | | |
| Sorbitan Monostearate | Sorbitan 60K | Food | ADM arkady | 3 | | |
| Magnesium stearate | | USP | whittakar | 0.6 | | |
| Purified Talc | | USP/NF | whittakar | 0.2 | | |
| Colloidal silicon dioxide | Syfloid 244F | USP/NF | Grace | 0.1 | | |
| Cherry flavor Spray dried | | | David Michael & Co. | 2.5 | | |
| Micronlsed Sucralose Powder | Splenda | NF | Mcneil | 0.2 | | |

The present invention has been described above with reference to a number of exemplary embodiments and examples. It should be appreciated that the particular embodiments shown and described herein are illustrative of the invention and its best mode and are not intended to limit in any way the scope of the invention as set forth in the claims. For example, although the composition, system, and method have been described in detail in connection with a tablet including zinc, the composition may alternatively be applied to an oral membrane via a dry powder and may include additional or alternative active ingredients. Those skilled in the art having read this disclosure will recognize that changes and modifications may be made to the exemplary embodiments without departing from the scope of the present invention. Further, though reference is made to “substances”, “agents”, and “ingredients”, skilled artisans will appreciate that these terms can be used interchangeably. Accordingly, these and other changes or modifications are intended to be included to be within the scope of the present invention, as expressed in the following claims.

1. A composition for treating symptoms associated with colds, the composition comprising:
an effective amount of an agent comprising a plurality of zinc salts, configured to reduce a severity of a cold symptom;
an inert agent; and
a dissolution agent,
wherein the composition is in the form of a dissolvable tablet.

2. The composition of claim 1, wherein the plurality of zinc salts comprises zinc acetate and zinc gluconate.

3. The composition of claim 1, wherein the agent comprising zinc comprises a homeopathic dose of zinc.

4. The composition of claim 1, further comprising a sweetener.

5. The composition of claim 4, wherein the sweetener comprises manitol.

6. The composition of claim 4, wherein the sweetener comprises sucralose.

7. The composition of claim 1, further comprising a thickener.

8. The composition of claim 7, wherein the thickener comprises cellulose.

9. The composition of claim 1, further comprising a permeation enhancer.

10. The composition of claim 1, further comprising a preservative.

11. The composition of claim 9, wherein the preservative comprises polysorbate 80.

12. The composition of claim 1, wherein the dissolution agent comprises a material selected from the group consisting of polyvinyl pyrolidone, sodium lauryl sulphate, and sodium starch glycolate.

13. The composition of claim 1, further comprising a texture agent.

14. The composition of claim 13, wherein the texture agent comprises a material selected from the group consisting of colloidal silicon dioxide; crosspovidone, magnesium stearate, and sorbitan monostearate.

15. The composition of claim 1, wherein the composition comprises about 1% to about 10 wt % zinc and about 40 wt % to about 60 wt % sweetener agent.

16. A method of forming a cold relief formula, the method comprising the steps of:
admixing active ingredients comprising a plurality of zinc salts and an inert substance to form an admixture; and
adding a dissolution agent to the admixture to form a cold remedy composition.

17. The method of claim 16, further comprising the step of forming the cold remedy composition into a tablet.

18. A method of relieving symptoms associated with a cold, the method comprising the steps of:
providing a quick-dissolving tablet comprising a plurality of zinc salts; and
applying the formula to an oral membrane.

19. A composition for reducing a duration and/or severity of a cold, the composition comprising:
about 1 to about 5 wt % of a first zinc salt;
about 1 to about 5 wt % of a second zinc salt;
about 0.01 to about 5 wt % of a dissolution agent;
a texturing agent; and
a preservative.

20. The composition of claim 19, wherein said dissolution agent is a compound selected from the group consisting of sodium lauryl sulfate, sodium starch glycolate, and polyvinyl pyrolidone.

21. The composition of claim 19, wherein the first zinc salt comprises zinc acetate.

22. The composition of claim 19, wherein the second zinc salt comprises zinc gluconate.
